(19) United States
(12) Patent Application Publication
(10) Pub. No.: US 2023/0357329 A1
(43) Pub. Date: Nov. 9, 2023

(54) COMBINED USE OF NANOBODY, CYCLODEXTRIN AND QUERCETIN FOR PROTECTION FROM ENVELOPED VIRUSES

(71) Applicant: VIRON, INC., Boston, MA (US)

(72) Inventor: Bhanu Pratap Jena, Bloomfield Hills, MI (US)

(73) Assignee: VIRON, INC., Boston, MA (US)

(21) Appl. No.: 18/222,784

(22) Filed: Jul. 17, 2023

Related U.S. Application Data
(63) Continuation-in-part of application No. 17/890,754, filed on Aug. 18, 2022.
Application No. 17/890,754 is a continuation-in-part of application No. 17/472,604, filed on Sep. 11, 2021.
Application No. 17/472,604 is a continuation-in-part of application No. 17/207,250, filed on Mar. 19, 2021.
(60) Provisional application No. 63/235,772, filed on Aug. 22, 2021.
(60) Provisional application No. 63/294,067, filed on Dec. 27, 2021.
(60) Provisional application No. 63/308,782, filed on Feb. 10, 2022.
(60) Provisional application No. 63/029,458, filed on May 23, 2020.
(60) Provisional application No. 63/019,312, filed on May 2, 2020.
(60) Provisional application No. 63/235,772, filed on Aug. 22, 2021.
(60) Provisional application No. 63/396,040, filed on Aug. 8, 2022.

(51) Int. Cl. C07K 14/165; A61K 31/724; A61K 31/7048; A61K 33/30

(52) U.S. Cl. CPC C07K 14/165; A61K 31/724; A61K 31/7048; A61K 33/30; C07K 2317/569

(57) ABSTRACT

Disclosed are engineered nanobodies against different target antigens such as the spike protein of the SARS-CoV-2 virus and or to the ACE2 receptor at the host cell surface, binds to the virus or the ACE2 receptor at the host cell plasma membrane, preventing entry of the SARS-CoV-2 virus.

### APPLICATION CROSS-REFERENCE

The instant application is a continuation-in-part of U.S. patent application Ser. No. 17/890,754, filed on Aug. 18, 2022, which claims the benefit of U.S. Provisional Patent Application Ser. No. 63/235,772, filed on Aug. 22, 2021, U.S. Provisional Patent Application Ser. No. 63/294,067, filed on Dec. 27, 2021, and U.S. Provisional Patent Application Ser. No. 63/308,782, filed on Feb. 10, 2022, and which is also a continuation-in-part of U.S. patent application Ser. No. 17/472,604, filed on Sep. 11, 2021, which is a continuation-in-part of U.S. patent application Ser. No. 17/207,250, filed on Mar. 19, 2021, and which claims the benefit of U.S. Provisional Patent Application Ser. No. 63/029,458, filed May 23, 2020, U.S. Provisional Patent Application Ser. No. 63/019,312, filed May 2, 2020, and U.S. Provisional Patent Application Serial No. 63/235,772, filed on Aug. 22, 2021. The instant application further claims priority to U.S. Provisional Patent Application Ser. No. 63/396,040, filed Aug. 8, 2022. The entirety of all the above referenced are hereby incorporated by reference.

### BACKGROUND

### TECHNICAL FIELD

Embodiments of the invention generally fall into the category of suppression or prevention of enveloped viral infection. In some embodiments there is provided a formulation of engineered nanobodies combined with Hydroxypropyl-β-Cyclodextrin and the flavonoid quercetin administered in a nasal/pulmonary route to treat infection with or exposure to an enveloped virus in a therapeutic or prophylactic fashion.

### DISCUSSION OF ART

Viruses enter hosts via the epithelium. The cell plasma membrane of skin and lung epithelia is the first line of defense and when breached, serves as the portal for viral entry into hosts. The entry of pathogenic viruses into the host cell could be prevented by using nanobodies such as against SARS-CoV-2 and or small molecules which have been demonstrated to be safe for human use.

Studies in the past two decades report the various cell membrane binding and entry mechanisms utilized by viruses to infect. Irrespective of the different mechanisms involved in viral entry into host cells, the initiating critical process is binding of the virus to the cell plasma membrane. Without binding of virus to the cell plasma membrane, there would be no viral entry into the host.

A multitude of studies have established that binding of viruses to the cell plasma membrane is subjected to the presence of docking sites or receptors and their regulation by membrane lipid composition and distribution such as the establishment of domains called rafts. A recent study involving cellular membrane biogenesis, demonstrates that changes in composition of membrane cholesterol, impacts both the chemistry and distribution of plasma membrane proteins and lipids, impacting cell function. That study reported that cells exposed to an increasing concentration of methyl beta cyclodextrin (M-βCD) to deplete cholesterol from the cell plasma membrane demonstrate loss in the uptake of phosphotidyl serine by the cell plasma membrane, while the uptake of phosphatidylethanolamine remain unchanged. Similarly, the loss of cholesterol from the cell plasma membrane resulted in the depletion of membrane fusion proteins such as syntaxin and SNAP25 from the plasma membrane suggesting altered membrane fusogenicity. Therefore changes to the chemistry of the epithelial cell plasma membrane via depletion of sterols/cholesterol by cyclodextrins (CDs), could dictate both the binding of a virus at a cell plasma membrane, and influence both the efficacy and potency of its entry into the host cell.

Further studies demonstrated that depletion of plasma membrane cholesterol in host cells using M-βCD, significantly reduces entry of the pseudorabies and vaccinia virus into cells. Additional studies demonstrated that HIV infectivity is critically dependent on cholesterol. Cholesterol microdomains, called lipid ‘rafts’, have been suggested in the cellular entry or infection of HIV, its assembly, and its release from infected cells. Studies further report that plasma membrane cholesterol is also required for a wide range of both bacterial and yeast infections. Furthermore, the literature illustrates that a high-cholesterol diet impairs pulmonary host defense against gram-negative bacteria and Mycobacterium tuberculosis. Taken together, these results support that CD-mediated depletion of plasma membrane cholesterol in epithelial cells (i.e., skin, nasal passage, and lung epithelia) in humans, using topological application, aerosol spray and nebulization, will mitigate both viral entry and secondary bacterial and yeast infections.

CDs are a family of cyclic oligosaccharides constituted of a macrocyclic ring of glucose subunits joined by α-1,4 glycosidic bonds. CDs are used for improving the water-solubility and bioavailability of a wide range of drugs. The U.S. Food and Drug Administration (FDA) has approved the use of cyclodextrins since 2001. Cyclodextrins were first employed in the food industry in the 1970s, and since they have been used as food additives for carrying food-related lipophiles such as vitamins, aromas, and colorants. βCD has also been used as a cholesterol-reducing agent in food of animal origin such as milk and eggs.

The first pharmaceutical patent related to CDs and pharmaceutical applicability as complexing agents is dated 1953. Currently, cyclodextrins are employed in pharmaceutical products primarily to increase water solubility of poorly soluble drug formulations and to enhance drug bioavailability. Pharmaceutical products containing CDs comprise nasal spray, oral solutions, solid dosage forms, ocular and dermal formulations, suppositories, and parenteral solutions. Currently, more than 40 pharmaceutical products containing CDs are available in the market worldwide, and many of them utilize βCD and its derivatives having higher water solubility such as HPβCD, MβCD, and SBEβCD. Most of the βCD are also approved by the European Medical Agency for all human administration pathways. CDs are used in tablets, aqueous parenteral solutions, nasal sprays, and eye drop solutions. Examples of the use of cyclodextrins in medicines on the European market are β-CD in cetirizine tablets and cisapride suppositories, γ-CD in minoxidil solution, and examples of the use of β-cyclodextrin derivatives are SBE-β-CD in the intravenous antimycotic voriconazole, HP-β-CD in the antifungal itraconazole, intravenous and oral solutions, and RM-β-CD in a nasal spray for hormone replacement therapy by 17β-estradiol. In Germany and Japan there are infusion products on the market, containing alprostadil (prostaglandin E1, PGE1) with α-CD. Cyclodextrins are currently not included in the European Commission Guideline on excipients in the label and package leaflet of medicinal products for human use.

Despite all the advancements above cataloged, there remains a need to treat infection with or exposure to an enveloped virus in a therapeutic or prophylactic fashion. In particular, there remains a need to stabilize or reduce the amount of virus present in a human or other animal.

### BRIEF SUMMARY OF THE INVENTION

In an embodiment of the invention, engineered nanobodies against different target antigens such as the spike protein of the SARS-CoV-2 virus and or to the ACE2 receptor at the host cell surface, binds to the virus or the ACE2 receptor at the host cell plasma membrane, preventing entry of the SARS-CoV-2 virus.

In another embodiment of the invention, Hydroxypropyl-Beta-Cyclodextrin (HPβCD), which has been affirmed as “Generally Recognized As Safe” (GRAS) by the U.S. Food and Drug Administration (FDA), is administered in different particle sizes in phosphate buffered solutions at concentration ranges of between one (1) and ten (10) per cent (%). The administration enables the extraction of cholesterol molecules to various extents from enveloped virus membranes, including that of SARS-CoV-2 and its variants, and from host cell membranes of human subjects thus altering their respective lipid and protein profiles and distribution and thus preventing enveloped virus entry into host cells of human subjects.

In another embodiment of the invention, a nanobody engineered against a virus or host cell membrane viral receptor is delivered via aerosol spray and/or nebulization. The nanobody is combined with either a uniform and/or a wide range of particle sizes of Hydroxypropyl-Beta-Cyclodextrin (HPβCD) in an aqueous phosphate-buffered saline solution at pH 5 to pH 7.5. The solution further contains 0.01% Benzylkonium chloride as a preservative. The solution may be prophylactically administered to protect the airways, including lungs of human subjects, from all enveloped virus infections including infections from SARS-CoV-2 and its variants.

In another embodiment the nanobody and Hydroxypropyl-Beta-Cyclodextrin (HPβCD) containing solution is administered via the pulmonary route for the prevention of the entry of SARS-CoV-2 or other enveloped viruses into lung epithelial cells.

In another embodiment, the flavonoid quercetin, a naturally occurring plant-based over the counter zinc ionophore that is “Generally Recognized As Safe” (GRAS) by the U.S. Food and Drug Administration (FDA), is administered in different particle sizes in phosphate buffered solutions at a concentration of 1-20 micrograms/ml to enable the cellular entry of zinc to protect host cells against viruses by inhibiting RNA binding, RNA synthesis, viral polyprotein cleavage, viral replication, and viral protease enzyme inactivation, among others.

In another embodiment, any of the described embodiments is administered to a patient for the prevention of both viral entry and replication.

In another embodiment, the engineered nanobody against the virus or the virus receptor at the host cell membrane, combined with Hydroxypropyl-Beta-Cyclodextrin (HPβCD) and quercetin in a aqueous phosphate buffered saline solution at pH 5 to pH 7.5 containing 0.01% Benzylkonium chloride as preservative is administered via aerosol spray or nebulization.

In another embodiment, the present invention is administered dermally either as a treatment or as a means of sanitization.

### DETAILED DESCRIPTION OF THE INVENTION

The invention provides the use of a formulation containing cyclodextrine, quercetin and zinc, at appropriate concentrations to mitigate infections by enveloped viruses like SARS-CoV-2, influenza and HIV/AIDS. While the different forms of cyclodextrin prevent the entry of coated virus into host cells by extracting and sequestering cholesterol molecules at the virus coat and at the host cell plasma membrane, the natural plant-based ionophore quercetin in the formulation, enables cellular entry of zinc, inhibiting viral replication by altering polymerase activity in the host cell. Without subscribing to or limiting to a single theory of operation, it is believed that use of cyclodextrin as a drug in phosphate buffered saline solutions, will allow the extraction of cholesterol molecules from enveloped virus membranes and the host cell membrane, altering their respective lipid and protein composition and distribution, thus preventing virus entry into host cells. The additional use of quercetin, a naturally occurring plant-based over the counter zinc ionophore, enables the cellular entry of zinc to protect host cells against the virus by inhibiting RNA binding, RNA synthesis, viral polyprotein cleavage, viral replication, and viral protease enzyme inactivation, among others.

The invention provides the use of a formulation containing engineered nanobodies against different target antigens of enveloped viruses and host cell membrane, in combination with Hydroxypropyl-Beta-Cyclodextrin (HPβCD) and the flavonoid quercetin, at appropriate concentrations to mitigate infections by enveloped viruses like SARS-CoV-2 and influenza, when administered via the nasal/pulmonary route.

Nanobodies are a subclass of antibodies composed of a single polypeptide chain with versatile molecular binding scaffolds found in the camel species, as opposed to the large y-shaped conventional antibodies found in other mammalian species including humans.

Without limitation to a single individual theory of operation, nanobodies may be engineered against different target antigens. For example, a nanobody, using conventional techniques, may be engineered against the spike protein of the SARS-CoV-2 virus and/or to the ACE2 receptor. The nanobody binds to the virus surface or the ACE2 receptor of the host cell preventing viral entry, the HPβCD in the formulation serves to prevent the entry of the enveloped virus into host cells by extracting and sequestering cholesterol molecules at the virus envelop and at the host cell plasma membrane.

However, if some viruses have entered the host cell, the natural plant-based ionophore quercetin in the formulation, enables cellular entry of zinc, inhibiting viral replication by altering polymerase activity in the host cell. Hence, engineered nanobodies in combination with HPβCD and quercetin, serve both as prophylactic treatment and therapeutic drug.

Without subscribing to or limiting to a particular theory, it is thought such a mixture will provide long term protection from targeted viruses and their variants. In some embodiments variable domains of the camelidaie variable domain (VHH) nanobodies are humanized to target and bind to one or more domains of one or more target viruses. In some embodiments protection from viral infection may last from 6-8 months. In some embodiments a viral spike protein is targeted. Spike proteins are readily identifiable from known motifs and are available through databases such as those run by the U.S. National Institutes of Health and the American Chemical Society. Nanobodies targeted to multiple regions in the spike protein bind and prevent entry of the virus into the host cell, thus providing protection from multiple viral variants.

Benzalkonium chloride widely used as a preservative in nasal sprays and nebulization, has been reported to cause sinonasal mucosal injury, nasal squamous metaplasia, ciliary dysmotility, genotoxicity, and other adverse effects. Data also suggests the toxic effects of phenylcarbinol, another commonly used preservative. Despite this evidence, these preservatives continue to be used at higher concentrations even in over-the-counter preparations. Acidification (pH 2.5) of nasal, inhalable, and topical ophthalmic preparations have been demonstrated to maintain sterility without the need for preservatives. This approach of lowering the pH of the formulated CD and quercetin solutions to be used in wipes and aerosol sprays, precludes the use of harmful preservatives at higher concentrations, without compromising sterility of the formulation. Therefore, either low concentration of benzalkonium chloride and or low pH formulations are prepared for use.

Natural CDs such as αCD, βCD, and γCD are hydrophilic in aqueous solutions, however they tend to self-assemble and form complexes. To overcome this limitation, soluble βCD derivatives such as 2-hydroxypropyl-βCD (HPβCD) and sulfobutylether βCD sodium salt (SBEβCD), are preferred for use in aqueous pharmaceutical formulations. Inorganic acids such as phosphoric and citric acid induce CD solubilization.

Zinc is an essential trace element supporting growth, development, and immune health. Zinc is also known to protect against viruses by inhibiting RNA binding, RNA synthesis, viral polyprotein cleavage, viral replication, and viral protease enzyme inactivation. Zinc however needs to enter the host cell to protect against the virus. Quercetin, a naturally occurring plant-based over the counter zinc ionophore, enables the cellular entry of zinc to protect host cells against the virus. Furthermore, quercetin has shown therapeutic effects against influenza virus. Additionally, in silico modelling of the interactions between the SARS-CoV-2 viral spike protein and the epithelial cell angiotensin converting enzyme-2 (ACE2) protein, has identified quercetin from a database of 8,000 small molecule candidates of known drugs, metabolites, and natural products, as one of the top 5 most potent compounds for binding to the interface site, and disrupt initiation of viral infection.

The present invention utilizes FDA approved concentrations of CDs (1-5%) and quercetin (8-24 micrograms mL⁻¹ ) in buffered solutions to retain both high solubility and sterility. Mode of administration may be through aerosol spray and/or nebulization, and topical application on body surface using a water-based solution adsorbed to paper, cellulose or fabric. The topical application on body surfaces will including the face and neck, to mitigate envelope virus (such as SARS-CoV-2, influenza, and HIV), bacteria and fungus infections.

In a particular embodiment, the formulation is adapted for use and introduction to a subject by dispersal, utilizing any known method of measured nasal sprays or inhalers. The formulation is introduced in form of an aqueous phosphate buffered saline pH 5-7.5 solution, containing 0.01% Benzylkonium chloride as preservative and different concentrations of the active ingredients: 1-5% cyclodextrin; and the flavonoid quercetin, a naturally occurring zinc ionophore at a concentration of 8-24 μg ml. Depending on requirement, pH 2.5 citrate buffered aqueous may also be used, where the low pH serves as a preservative and a solvent for cyclodextrin.

In another embodiment, administration of cyclodextrin alone is followed by quercetin and zinc administration (alone or combined), in a water based soluble formulation that prevents both viral entry and replication in host cells, such as the nasal epithelial cells and that of the lung epithelia. The formulation may be administered as an aerosol spray or nebulization. A particular embodiment contemplates an aqueous phosphate buffered saline pH 7.5 solution containing 0.01% Benzylkonium chloride as preservative, will be used to protect the airways including lungs from all coat virus infections. In still another embodiment the pH may be citrate buffered at pH 2.5.

In additional embodiments the above formulations may be topically applied; for example, via oils, creams, emulsions and the like the formulations of which are known to those skilled in the art. Additional embodiments include application of the above solutions to both sides of cellulose masks. In such medicated masks, any airborne droplets containing the virus will be neutralized on contact with the medicated mask.

Suitable alterations to the above are readily apparent to those of skill in the art and naturally are encompassed and expressly contemplated. For example, normal manufacturing tolerances may induce variances from the above presented formulations without departing from the broader scope of this invention.

The effective dose and method of administration of a particular embodiment of the instant invention may vary based on the individual patient and stage of any present diseases (e.g., influenza, covid, HIV, other co-morbidities), as well as other factors known to those of skill in the art. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The exact dosage is chosen by an individual physician in view of a patient to be treated. Dosage and administration are adjusted to provide sufficient levels of embodiments of the instant invention to maintain the desired effect (e.g., elimination or reduction of enveloped virus particles or activity in a host). Additional factors that may be taken into account include the severity of any disease state, age, weight, and gender of the patient; diet, time and frequency of the administration, drug combination(s), reaction sensitivities, and tolerance/response to therapy.

Short acting pharmaceutical compositions are administered daily whereas long acting pharmaceutical compositions are administered every 2, 3 to 4 days, every week, or once every two weeks or more. Depending on half-life and clearance rate of the particular formulation, the pharmaceutical compositions of the instant invention may be administered once, twice, three, four, five, six, seven, eight, nine, ten or more times per day.

Normal dosage amounts for active ingredients may vary from approximately 1 to 100,000 micrograms, up to a total dose of about 10 grams, depending upon the route of administration. Desirable dosages include 250 μg, 500 μg, 1 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1 g, 1.1 g, 1.2 g, 1.3 g, 1.4 g, 1.5 g, 1.6 g, 1.7 g, 1.8 g, 1.9 g, 2 g, 3 g, 4 g, 5 g, 6 g, 7 g, 8 g, 9 g, and 10 g.

More specifically, the dosage of the active ingredients described herein are those that provides sufficient quantity to attain a desirable effect, including those above-described effects. Accordingly, the dose of the active ingredients preferably produces a tissue or blood concentration of both about 1 to 800 μM. Preferable doses produces a tissue or blood concentration of greater than about 10 μM to about 500 μM. Preferable doses are, for example, the amount of active ingredients required to achieve a tissue or blood concentration or both of 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 110 μM, 120 μM, 130 μM, 140 μM, 150 μM, 160 μM, 170 μM, 180 μM, 190 μM, 200 μM, 220 μM, 240 μM, 250 μM, 260 μM, 280 μM, 300 μM, 320 μM, 340 μM, 360 μM, 380 μM, 400 μM, 420 μM, 440 μM, 460 μM, 480 μM, and 500 μM. Although doses that produce a tissue concentration greater than 800 μM are not necessarily preferred, they are envisioned and can be used with some embodiments of the present invention. A constant infusion of embodiments of the invention can be provided so as to maintain a stable concentration of the therapeutic agents.

Finally, the written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

The pharmacologically active compounds of this invention can be processed in accordance with conventional methods of galenic pharmacy to produce medicinal agents for administration to patients (e.g., mammals including humans).

As used herein the term “sequence” explicitly contemplates DNA, cDNA, RNA and resulting peptide chains encoded thereby in both sense and antisense directions. To know one is to know the others via the standard rules of complementarity and codon encoding as exemplified in standardized DNA, RNA, and amino acid codon tables.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

Since certain changes may be made in the above-described invention, without departing from the spirit and scope of the invention herein involved, it is intended that all of the subject matter of the above description shown in the accompanying drawings shall be interpreted merely as examples illustrating the inventive concept herein and shall not be construed as limiting the invention.

What is claimed is:

1. A composition comprising:
a nanobody;
a cyclodextrin;
a zinc ionophore;
a zinc-containing compound; and, benzalkonium.

2. The composition of claim 1 wherein the nanobody is engineered to attach to a viral protein.

3. The composition of claim 2 wherein the viral protein is a viral spike protein.

4. The composition of claim 3 wherein the viral spike protein is a coronavirus spike protein.

5. The composition of claim 1 wherein the nanobody is engineered to attach to a host cell receptor.

6. The composition of claim 5 wherein the host cell receptor is ACE2.

7. The composition of claim 1 wherein the zinc ionophore is quercetin.

8. The composition of claim 7, wherein:
the quercetin is at a concentration of 8 μg mL⁻¹; and
the zinc containing compound is zinc chloride at a concentration of 1 mg mL⁻¹.

9. The composition of claim 1 wherein the composition is buffered between pH 2.5-7.5

10. The composition of claim 1 wherein the cyclodextrin is HPβCD at a concentration between 1-10%.

11. The composition of claim 1 wherein the composition is atomized and administered to the airways of a subject.

12. A composition, comprising:
a nanobody engineered to attach to a host cell membrane protein;
HPμCD at a concentration of 1-5%;
quercetin at a concentration of 8 μg mL⁻¹;
zinc chloride at a concentration of 1 mg mL¹; and, benzalkonium at a concentration of 0.01%;
wherein the solution is either citrate buffered at a pH of 2.5 or phosphate buffered at a pH of 7.5.

13. The composition of claim 11 wherein the composition is atomized and administered to the airways of a subject.

14. The composition of claim 13 wherein the administration occurs in two steps, wherein the nanobody and cyclodextrin is first administered and the quercetin and zinc is administered second.

15. The composition of claim 12 wherein the composition is suspended in an aqueous solution.

16. A method of treating a subject at risk of contracting a viral infection, caused by an enveloped virus, the method comprising the step of:
administering to the subject a composition comprising a nanobody, a cyclodextrin, a zinc ionophore; a zinc-containing compound, and benzalkonium.

17. The method according to claim 16, wherein:
the nanobody is engineered to attach to a viral protein.

18. The method according to claim 17, wherein:
the viral protein is a viral spike protein.

19. The method according to claim 18, wherein:
the viral spike protein is a coronavirus spike protein.

20. The method according to claim 18, wherein:
the zinc ionophore is quercetin.
